(19) United States
(12) Patent Application Publication
(10) Pub. No.: US 2009/0182428 A1
(43) Pub. Date: Jul. 16, 2009

(54) FLANGED INTERBODY DEVICE

(75) Inventors: John W. McClellan, III, Omaha, NE (US); Sarah Stamm, Omaha, NE (US)

Correspondence Address: Rahman LLC, 10025 Governor Warfield Parkway, Suite 110, Columbia, MD, 21044, US

(73) Assignee: Custom Spine, Inc., Parsippany, NJ (US)

(21) Appl. No.: 12/015,024

(22) Filed: Jan. 16, 2008

(51) Int. Cl. A61F 2/44

(52) U.S. Cl. 623/17.16; 623/17.11

(57) ABSTRACT

An interbody implant including a first lateral portion, a second lateral portion positioned opposite to the first lateral portion, a top wall having a plurality of holes, a bottom wall positioned opposite to the top wall, and teeth positioned on the top wall and the bottom wall. The top wall and the bottom wall are attached to the first lateral portion and the second lateral portion. The first lateral portion further includes at least one flange surface and the second lateral portion further includes an opening. The at least one flange surface may be configured to couple to a peripheral wall of the vertebral body and may be adapted to provide at least one of a torsional property, an axial property, and a shear property to the implant. The teeth may be adapted to provide a mechanical interlock between the implant and vertebral endplates of the vertebral body.

### BACKGROUND

1. Technical Field

The embodiments herein generally relate to medical devices, and more particularly, to a flanged interbody device used during orthopedic surgeries.

2. Description of the Related Art

Anterior lumbar interbody fusion (ALIF), posterior lumbar interbody fusion (PLIF), and transforaminal lumbar interbody fusion (TLIF) are common spinal fusion procedures for fusing and stabilizing vertebrae. In these procedures, interbody spacers are placed within the intervertebral disc space. They are responsible for transmitting load across the disc space from one vertebra to the other. The spacer serves as a temporary column or structural support until fusion occurs. The loads across the interbody spacer include the weight of the person and any load being carried by the person.

Peripheral walls of the vertebrae are the strongest bones on vertebral endplates whereas subchondral bone (e.g., bone beneath cartilage) is the soft and weaker bone. Anterior column support may fail if the interbody spacer subsides through the vertebral endplates. Also, if the interbody spacer subsides the entire load is transferred to soft subchondral bone. Consequently, this may lead to increased pain and potentially neurologic complications. It may also lead to malunion, which is a successful fusion but with the vertebrae in nonanatomic alignment or suboptimal alignment. Furthermore, it may also lead to pseudarthrosis or failure of fusion.

Interbody spacers are typically available as threaded cylinders, screws, etc. Surface area is important to controlling postoperative pain and achieving successful fusion. Conventional implants tend to provide a limited amount of surface area. Conventional implants also generally do not include any supporting structure which can prevent decoupling of the implant from the vertebrae, which gives rise to subsidence of the implants. Some implants also suffer from the disadvantage of involving piercing and tapping of vertebral endplates for insertion. Additionally, restoration of natural curvature of the spine is also very difficult. Most implants are available in different sizes (e.g., longer and wider implants). The longer implants may be clinically specified but the wider implants are not desirable as the increased width involves more of facet scissoring which leads to a decrease in stability. Accordingly, there remains a need for a new interbody device to prevent subsidence while increasing torsional stability.

### SUMMARY

In view of the foregoing, an embodiment herein provides a flanged interbody device to prevent subsidence while increasing torsional stability. The flanged interbody device includes an implant to be inserted in a vertebral body. The implant includes a first lateral portion, a second lateral portion, a top wall, a bottom wall and teeth positioned on the top wall and the bottom wall. The second lateral portion is positioned opposite to the first lateral portion. The top wall and the bottom wall are attached to the first lateral portion and the second lateral portion. The bottom wall is positioned opposite to the top wall. The first lateral portion further includes at least one flange surface and the second lateral portion further includes an opening. The top wall further includes a plurality of holes. The implant may include a plurality of cuts positioned between the first lateral portion and the second lateral portion.

The teeth may be adapted to provide a mechanical interlock between the implant and vertebral endplates of the vertebral body. The plurality of holes and the plurality of cuts may be dimensioned and configured to receive bone graft material. The plurality of holes may be dimensioned and configured to receive pre-insertion bone graft material and the plurality of cuts may be dimensioned and configured to receive post-insertion bone graft material. The opening on the second lateral portion may be dimensioned and configured to accommodate an insertion tool to place the implant in the vertebral body. The at least one flange surface may be configured to couple to a peripheral wall of the vertebral body and may be adapted to provide at least one of a torsional property, an axial property, and a shear property to the implant.

Another aspect provides an apparatus to stabilize a vertebral body. The vertebral body includes a peripheral wall and a subchondral bone. The apparatus includes a first interbody implant which includes a first lateral portion having a flattened configuration, a second lateral portion positioned opposite to the first lateral portion, a top wall, a bottom wall positioned opposite to the top wall, and at least one cut positioned between the first lateral portion and the second lateral portion. The second lateral portion includes a tapered configuration with a width smaller than the width of the first lateral portion. The top wall and the bottom wall are attached to the first lateral portion and the second lateral portion.

The first lateral portion further includes a top flange surface and a bottom flange surface. The top wall further includes top teeth and at least one hole, and the bottom wall further includes bottom teeth. The top flange surface and the bottom flange surface are coupled to the peripheral wall of the vertebral body and adapted to divert vertebral forces to the peripheral wall of the vertebral body and from the subchondral bone. The vertebral forces may be at least one of a torsional force, an axial force, or a shear force.

The apparatus may include a second interbody implant placed in the vertebral body from a direction opposite to that of the first interbody implant. The top teeth and the bottom teeth may be adapted to provide mechanical interlock between the first interbody implant and the vertebral body. The at least one hole and the at least one cut may be dimensioned and configured to receive bone graft material. The apparatus may include an opening positioned on the second lateral portion. The opening may be adapted to accommodate an insertion tool to place the first interbody implant in the vertebral body.

Another embodiment provides a method of performing a surgical procedure that includes inserting a first interbody implant in a vertebral body, engaging teeth of the first interbody implant to endplates of the vertebral body, inserting a second interbody implant into the vertebral body from a direction opposite to that of the first interbody implant, positioning an inserter tool through an opening of the first interbody implant for placement of the first interbody implant in the vertebral body, compressing the vertebral body against the first interbody implant and inserting bone grafting material in the first interbody implant.

The vertebral body includes a peripheral wall and the endplates. The first interbody implant includes a flanged end opposite to a tapered end. A flange surface of the flanged end may be adapted to provide at least one of a torsional property, an axial property, or a shear property to the first interbody implant. Additionally, the first interbody includes outwardly protruding teeth positioned along a top wall and a bottom wall of the first interbody implant. The teeth may be adapted to provide mechanical interlock between the first interbody implant and the endplates of the vertebral body. The first interbody implant is dimensioned and configured based on a physical property of a vertebral segment of the vertebral body supported by the first interbody implant. The physical property may be at least one of a length, a surface area, or a lordosis. The direction may be at least one of an anterior direction, a posterior direction, or a lateral direction with respect to the vertebral body.

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### BRIEF DESCRIPTION OF THE DRAWINGS

The embodiments herein will be better understood from the following detailed description with reference to the drawings, in which:

FIG. 1A illustrates a front view of a flanged interbody device according to an embodiment herein;

FIG. 1B illustrates a top view of the flanged interbody device according to an embodiment herein;

FIG. 1C illustrates a side view of the flanged interbody device according to an embodiment herein;

FIG. 2A illustrates a schematic view of the flanged interbody device of FIGS. 1A through 1C inserted in the column area to a vertebral body in an ALIF position according to an embodiment herein;

FIG. 2B illustrates a schematic view of the flanged interbody device of FIGS. 1A through 1C inserted in the column area to a vertebral body in a TLIF position according to an embodiment herein;

FIG. 2C illustrates a schematic view of the flanged interbody device of FIGS. 1A through 1C inserted in the column area to a vertebral body in a lateral implantation position according to an embodiment herein; and

FIG. 3 illustrates a process flow illustrating a method of performing a surgical procedure according to an embodiment herein.

### DETAILED DESCRIPTION OF PREFERRED EMBODIMENTS

The embodiments herein and the various features and advantageous details thereof are explained more fully with reference to the non-limiting embodiments that are illustrated in the accompanying drawings and detailed in the following description. Descriptions of well-known components and processing techniques are omitted so as to not unnecessarily obscure the embodiments herein. The examples used herein are intended merely to facilitate an understanding of ways in which the embodiments herein may be practiced and to further enable those of skill in the art to practice the embodiments herein. Accordingly, the examples should not be construed as limiting the scope of the embodiments herein.

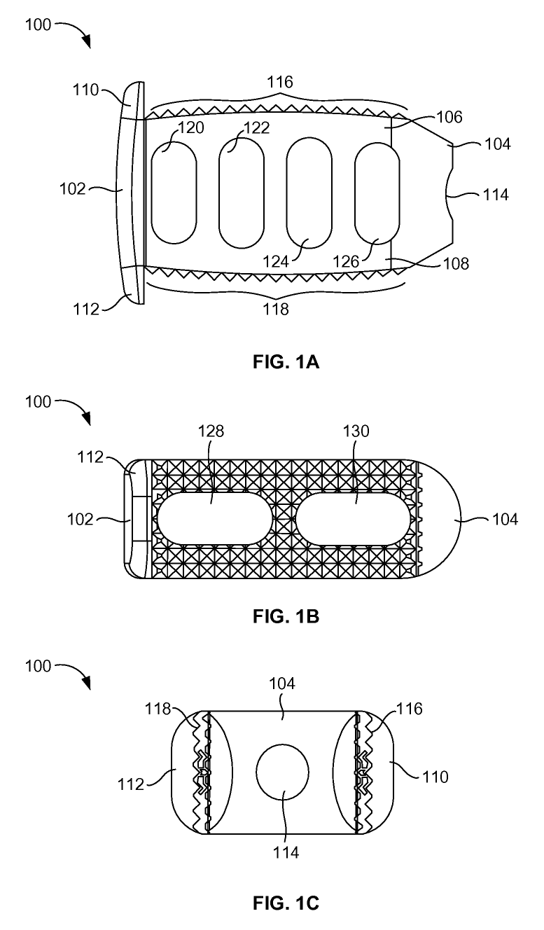

As mentioned, there remains a need for a new interbody device to prevent subsidence while increasing torsional stability. The embodiments herein achieve this by providing an interbody device that uses a flange to allow the lateral vertebral body to participate in load bearing and shear and torsional resistance. The device can be used in the anterior column (front of the vertebrae) or middle column (which may be the middle or anterior column). Referring now to the drawings and more particularly to FIG. 1A through FIG. 3, where similar reference characters denote corresponding features consistently throughout the figures, there are shown preferred embodiments.

FIGS. 1A through 1C illustrate a front view, top view, and a side view, respectively, of a flanged interbody device 100 according to an embodiment herein. The interbody device 100 includes a first lateral portion 102, a second lateral portion 104 positioned opposite to the first lateral portion 102, a top wall 106, and a bottom wall 108. Both the top wall 106 and the bottom wall 108 are attached to the first lateral portion 102 and as well as the second lateral portion 104. The second lateral portion 104 has a tapered configuration with a width smaller than a width of the first lateral portion 102.

The first lateral portion 102 further includes a top flange surface 110 and a bottom flange surface 112. The second lateral portion 104 includes a concave opening 114. The top wall 106 and the bottom wall 108 include outwardly protruding teeth 116, 118, respectively. Additionally, the interbody device 100 includes a plurality of cuts 120, 122, 124, 126 positioned between the lateral portions 102, 104, out of which cuts 122,124 are of same length and width and cuts 120, 126 are of same length and width. The top wall 106 includes two holes 128, 130 which are of uniform length and width. The top flange surface 110 and the bottom flange surface 112 of the implant 100 extend beyond the width of the implant 100.

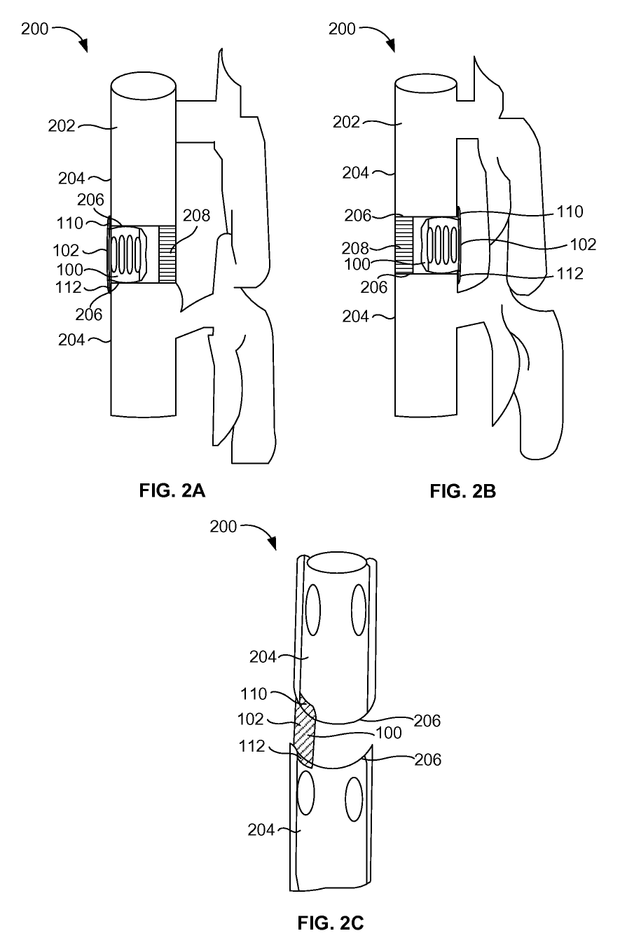

FIGS. 2A through 2C illustrate schematic views of the flanged interbody device 100 of FIGS. 1A through 1C inserted in a column area 200 to a vertebral body 202 in an ALIF position, a TLIF position, and a lateral implantation position, respectively. The vertebral body 202 includes two peripheral walls 204 and two endplates 206. FIGS. 2A through 2C further illustrate a second interbody implant 208. When the interbody device 100 is inserted into the vertebral body 202, the two flange surfaces 110, 112 of the interbody device 100 are placed on the peripheral walls 204 of the vertebral body 202. This prevents subsidence of the interbody device 100 into end plates 206 of the vertebral body 202 over time. The two flange surfaces 110, 112 improve torsional, axial, and shear properties of the interbody device 100 due to the teeth 116, 118, and thus provide added stability to the interbody device 100. The teeth 116, 118 penetrate the vertebral endplates 206 of the vertebral body 202 and thus provide a mechanical interlock between the interbody device 100 and the vertebral endplates 206. The mechanical stability afforded by the teeth 116, 118 may minimize the risk of postoperative expulsion of the interbody device 100.

The cuts 120, 122, 124, 126 and the holes 128, 130 are dimensioned and configured for insertion of bone graft material. The holes 128, 130 may be dimensioned and configured for pre-insertion bone graft material packing. Bone graft material may be inserted through the holes 128, 130 of the interbody device 100 before the interbody device 100 is inserted into the vertebral body 202. The cuts 120, 122, 124, 126 may be adapted for post-insertion bone graft material packing. Bone graft material may be inserted through the cuts 120, 122, 124, 126 of the implant 100 after the interbody device 100 is inserted into the vertebral body 202. The opening 114 may serve as a position to accommodate insertion tools (e.g., not shown) to be placed for repositioning of the interbody device 100 in the column area 200 in the vertebral body 202. The interbody device 100 may be used as a stand-alone implant or may be used as an adjunct to the second interbody implant 208 to reduce the risk of subsidence or provide improved torsional resistance to the other interbody supports.

The interbody device 100 may be used to augment conventional interbody devices. As depicted in FIGS. 2A through 2C, the interbody device 100 can be inserted in anterior, posterior, and/or lateral directions. The interbody device 100 can be inserted anteriorly and the second interbody implant 208 can be inserted posteriorly (e.g., as shown in FIG. 2A). The interbody device 100 can be inserted posteriorly and the second interbody implant 208 can be inserted anteriorly (e.g., as shown in FIG. 2B). The interbody device 100 can also be inserted laterally (e.g., as shown in FIG. 2C).

Preferably, the interbody device 100 is placed by a surgeon at the periphery of the vertebral endplates 206. The interbody device 100 may compress the vertebral body 202 against the interbody device 100 providing increase in stiffness/strength of construct. The ALIF approach (e.g., as illustrated in FIG. 2A) of inserting the interbody device 100 may prevent facet scissoring if used in conjunction with another interbody support (e.g., the second interbody implant 208). Moreover, the interbody device 100 is dimensioned and configured based on a physical property (e.g., at least one of a length, a surface area, and a lordosis) of a vertebral segment of the vertebral body 202 supported by the interbody device 100. Furthermore, variable sizes of the device 100 allow the surgeon to adjust the height of the interbody device 100 as well as the surface area supported. Thus, the lordosis of segments of the vertebral body 202 can be detached and available graft areas can be maximized.

The interbody device 100 also decreases the risk of subsidence by allowing the peripheral walls 204 of the vertebrae 202 to carry a partial load (e.g., through the flange surfaces 110, 112). As the interbody device 100 is placed on the peripheral walls 204, the flange surfaces 110, 112 allow the lateral vertebral wall 204 to participate in preventing subsidence of the interbody device 100. The lateral vertebral wall 204 also carries part of the axial load rather than soft subchondral endplate bone 206 carrying the entire load. This significantly decreases the risk of subsidence and should directly translate into less postoperative pain and less risk of pseudarthrosis. The resistance provided by the teeth 116, 118 will no longer be the only resistance to shear or torsional forces. The flange surfaces 110, 112 also participate in resisting torsional, axial and shear forces.

The second interbody implant 208 may be placed in a direction opposite to that of the interbody device 100 in the vertebral body 202. The direction may include at least one of an anterior direction, a posterior direction, and a lateral direction with respect to the vertebral body 202. The interbody device 100 with the flange surfaces 110, 112 also allows the surgeon to use the second interbody implant 208. Thus, the load bearing capacity of the interbody device 100 is significantly improved by diverting vertebral forces (e.g., at least one of a torsional force, an axial force, and a shear force) from traveling entirely through the top and bottom flange surfaces 110, 112, respectively of the interbody device 100 into the endplate 206 and the soft subchondral bone.

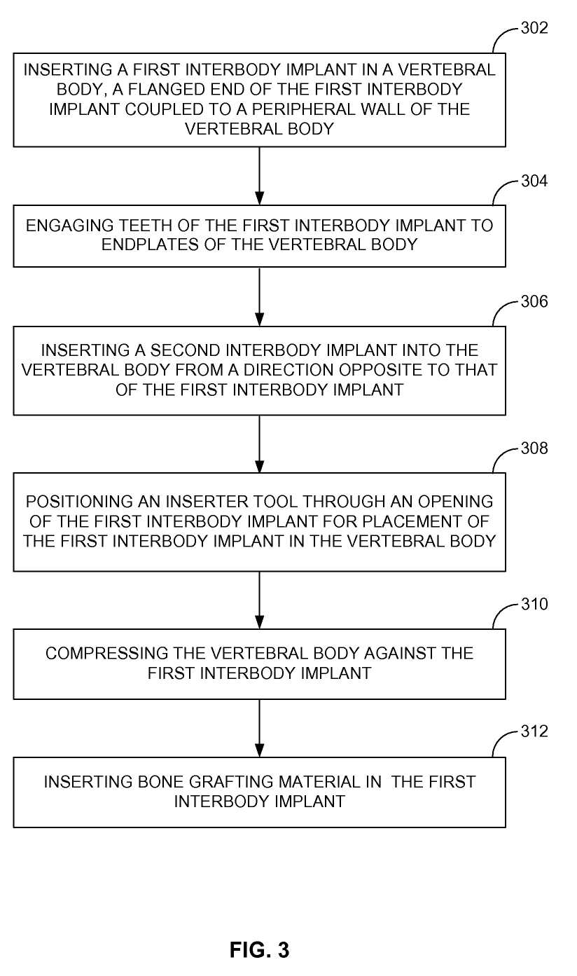

FIG. 3, with reference to FIGS. 1A through 2C, illustrates a process flow diagram illustrating a method of performing a surgical procedure according to an embodiment herein, wherein the method comprises inserting (302) a first interbody implant (e.g., the interbody device 100) in a vertebral body 202, engaging (304) teeth 116, 118 of the first interbody implant 100 to endplates 206 of the vertebral body 202, inserting (306) a second interbody implant 208 into the vertebral body 202 from a direction opposite to that of the first interbody implant 100, positioning (308) an inserter tool (e.g., not shown) through an opening 114 of the first interbody implant 100 for placement of the first interbody implant 100 in the vertebral body 202, compressing (310) the vertebral body 202 against the first interbody implant 100, and inserting (312) bone grafting material in the first interbody implant 100.

In step 302, the first interbody implant 100 is inserted into the vertebral body 202. The flange surfaces 110, 112 are coupled to the peripheral walls 204 of the vertebral body 202. In step 304, the teeth 116, 118 of the first interbody implant 100 are engaged to the endplates 206 of the vertebral body 202 (e.g., as illustrated in FIGS. 2A through 2C). The teeth 116, 118 are adapted to provide a mechanical interlock between the first interbody implant 100 and the endplates 206 of the vertebral body 202. In step 306, the second interbody implant 208 is inserted into the vertebral body 202 from a direction (e.g., at least one of an anterior direction, a posterior direction, and a lateral direction with respect to the vertebral body 202) opposite to that of the first interbody implant 100 (e.g., as illustrated in FIGS. 2A through 2C). In step 308, positioning (308) the inserter tool occurs through the opening 114 of the first interbody implant 100 for placement of the first interbody implant 100 in the vertebral body 202. In step 310, the vertebral body 202 is compressed against the first interbody implant 100. In step 312, bone grafting material is inserted in the first interbody implant 100 (e.g., through the cuts 120, 122, 124, 126 and the holes 128, 130). The bone grafting material may be inserted through the holes 128, 130 of the first interbody implant 100 before the interbody implant 100 is inserted into the vertebral body 202. Moreover, the bone grafting material may be inserted through the cuts 120, 122, 124, 126 after the interbody implant 100 is inserted into the vertebral body 202.

The interbody device 100 allows the peripheral walls 204 of the vertebral body 202 or the most peripheral portion of the vertebral body 202 to take a significant force from the implant 100. Moreover, the interbody device 100 permits a lateral portion of the vertebral body 202 to share in the forces through the interbody device 100. Additionally, the interbody device 100 may be used or inserted from any direction that the interbody implant 206 may be placed. The interbody device 100 further increases an axial, a shear, and a torsional stability, which, in turn, provides significant clinical benefit to postoperative patients. The interbody device 100 resists subsidence and provides improved torsional resistance thus resulting in less risk of subsidence and ultimately a safer and more comfortable recovery from interbody fusion surgery.

Generally, the interbody device 100 comprises a first lateral portion 102, a second lateral portion 104, a top wall 106, and a bottom wall 108. The second lateral portion 104 is positioned opposite to the first lateral portion 102. The top wall 106 and the bottom wall 108 are attached to the first lateral portion 102 as well as the second lateral portion 104. The bottom wall 108 is positioned opposite to the top wall 106. The first lateral portion 102 further includes at least one flange surface 110 and the second lateral portion 104 further includes an opening 114. The top wall 106 further comprises at least one hole 128, 130. Both the top wall 106 and the bottom wall 108 include teeth 116, 118, respectively.

The foregoing description of the specific embodiments will so fully reveal the general nature of the embodiments herein that others can, by applying current knowledge, readily modify and/or adapt for various applications such specific embodiments without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. Therefore, while the embodiments herein have been described in terms of preferred embodiments, those skilled in the art will recognize that the embodiments herein can be practiced with modification within the spirit and scope of the appended claims.

What is claimed is:

1. An implant to be inserted in a vertebral body, said implant comprising:
a first lateral portion comprising at least one flange surface;
a second lateral portion positioned opposite to said first lateral portion and having an opening;
a top wall attached to said first lateral portion and said second lateral portion, said top wall comprising a plurality of holes;
a bottom wall positioned opposite to said top wall and attached to said first lateral portion and said second lateral portion; and
teeth positioned on said top wall and said bottom wall.

2. The implant of claim 1, further comprising a plurality of cuts positioned between said first lateral portion and said second lateral portion.

3. The implant of claim 1, wherein said at least one flange surface is configured to couple to a peripheral wall of said vertebral body.

4. The implant of claim 1, wherein said teeth are adapted to provide a mechanical interlock between said implant and vertebral endplates of said vertebral body.

5. The implant of claim 2, wherein said plurality of holes and said plurality of cuts are dimensioned and configured to receive bone graft material.

6. The implant of claim 5, wherein said plurality of holes are dimensioned and configured to receive pre-insertion bone graft material and said plurality of cuts are dimensioned and configured to receive post-insertion bone graft material.

7. The implant of claim 1, wherein said opening on said second lateral portion is dimensioned and configured to accommodate an insertion tool to place said implant in said vertebral body.

8. The implant of claim 1, wherein said at least one flange surface is adapted to provide at least one of a torsional property, an axial property, and a shear property to said implant.

9. An apparatus to stabilize a vertebral body, said vertebral body comprising a peripheral wall, wherein said apparatus comprises a first interbody implant comprising:
a first lateral portion having a flattened configuration and comprising a top flange surface and a bottom flange surface, said top flange surface and said bottom flange surface coupled to said peripheral wall of said vertebral body, wherein said top flange surface and said bottom flange surface are each adapted to divert vertebral forces to said peripheral wall of said vertebral body;
a second lateral portion positioned opposite to said first lateral portion and having a tapered configuration with a width smaller than a width of said first lateral portion;
a top wall attached to said first lateral portion and said second lateral portion, said top wall comprising top teeth and at least one hole;
a bottom wall positioned opposite to said top wall and attached to said first lateral portion and said second lateral portion, said bottom wall comprising bottom teeth; and
at least one cut positioned between said first lateral portion and said second lateral portion.

10. The apparatus of claim 9, further comprising a second interbody implant placed in said vertebral body from a direction opposite to that of said first interbody implant.

11. The apparatus of claim 9, wherein said top teeth and said bottom teeth are adapted to provide a mechanical interlock between said first interbody implant and said vertebral body.

12. The apparatus of claim 9, wherein said vertebral forces comprise at least one of a torsional force, an axial force, and a shear force.

13. The apparatus of claim 9, wherein said at least one hole and said at least one cut are dimensioned and configured to receive bone graft material.

14. The apparatus of claim 9, further comprising an opening positioned on said second lateral portion and adapted to accommodate an insertion tool, said insertion tool to place said first interbody implant in said vertebral body.

15. A method of performing a surgical procedure, said method comprising:
inserting a first interbody implant in a vertebral body, said vertebral body having a peripheral wall and endplates, wherein said first interbody implant comprises a flanged end opposite to a tapered end, and outwardly protruding teeth positioned along a top wall and a bottom wall of said first interbody implant, wherein said first interbody implant is dimensioned and configured based on a physical property of a vertebral segment of said vertebral body supported by said first interbody implant, and said flanged end of said first interbody implant is coupled to said peripheral wall of said vertebral body;
engaging said teeth of said first interbody implant to said endplates of said vertebral body; and
inserting a second interbody implant into said vertebral body from a direction opposite to that of said first interbody implant.

16. The method of claim 15, further comprising positioning an inserter tool through an opening of said first interbody implant for placement of said first interbody implant in said vertebral body.

17. The method of claim 15, further comprising compressing said vertebral body against said first interbody implant and inserting bone grafting material in said first interbody implant.

18. The method of claim 15, wherein said physical property comprises at least one of a length, a surface area, and a lordosis.

19. The method of claim 15, wherein said direction comprises at least one of an anterior direction, a posterior direction, and a lateral direction with respect to said vertebral body.

20. The method of claim 15, wherein a flange surface of said flanged end is adapted to provide at least one of a torsional property, an axial property, and a shear property to said first interbody implant, and wherein said teeth are adapted to provide a mechanical interlock between said first interbody implant and said endplates of said vertebral body.
